(19) United States
(12) Patent Application Publication
(10) Pub. No.: US 2017/0215830 A1
(43) Pub. Date: Aug. 3, 2017

(54) MEDICAL INSTALLATION, AND METHOD FOR CONTROLLING A MEDICAL APPARATUS THEREIN

(71) Applicant: Siemens Aktiengesellschaft, Muenchen (DE)

(72) Inventors: Andre Henning, Erlangen (DE); Stefan Popescu, Erlangen (DE)

(73) Assignee: Siemens Aktiengesellschaft, Muenchen (DE)

(21) Appl. No.: 15/485,633

(22) Filed: Apr. 12, 2017

Related U.S. Application Data
(63) Continuation of application No. 13/486,188, filed on Jun. 1, 2012.

(30) Foreign Application Priority Data
Jun. 1, 2011 (DE) 102011076885.8

(51) Int. Cl. A61B 6/00; A61B 5/08; G01R 33/567; A61N 5/10; A61B 5/00; A61B 6/04; A61B 5/0408; A61B 6/03

(52) U.S. Cl. CPC A61B 6/541; A61B 5/04085; A61B 5/0809; A61B 6/032; A61B 6/037; A61N 5/1068; A61B 6/4417; A61B 5/6892; A61B 6/0492; A61B 6/503; A61B 6/527; G01R 33/5673; A61B 2562/0214

(57) ABSTRACT

In a method to control a medical apparatus of an installation having: a contact device for a patient, at least one electrical potential sensor that can be coupled to the body of said patient is integrated into the contact device. A signal evaluation device is provided with measurement signals generated with the electrical potential sensor for evaluation. The medical apparatus is connected with the signal evaluation device, and measurement signals that relate to the breathing and/or cardiac activity of the patient are acquired with the at least one electrical potential sensor coupled to the body of said patient upon contact of the patient with the contact device. Trigger signals are generated with the signal evaluation device based on the measurement signals that relate to the breathing cycle and/or the cardiac cycle of the patient. Operation of the medical apparatus is controlled based on the trigger signals.

### RELATED APPLICATION

The present application is a continuation of U.S. application Ser. No. 13/486,188, filed Jun. 1, 2012. Applicant claims the benefit of the filing date of that prior application under 35 U.S.C. §120. The content of this prior application is incorporated herein by reference.

### BACKGROUND OF THE INVENTION

Field of the Invention

The present invention concerns a method to control a medical apparatus, in particular for generation of images, which are optimally free of movement artifacts, of a tissue of the patient who moves due to cardiac activity and/or breathing, and/or for radiation therapy of a tissue of the patient who is moving due to cardiac activity and/or breathing. The invention also concerns an installation with such a medical apparatus and a computer to execute such a method, as well as a non-transitory data storage medium embodying programming instructions (commands) to execute such a method.

Description of the Prior Art

In the examination of tissue of a patient with an imaging apparatus, for example with an x-ray computed tomography apparatus, multiple 2D x-ray projections of the tissue are acquired respectively from different projection directions, most often during advancement of the tissue of the patient relative to the x-ray acquisition system of the x-ray computed tomograph. The goal of the examination is the generation of qualitatively high-grade and relevant images of the tissue based on the 2D x-ray projections, which images frequently form the basis for a medical diagnosis.

If tissue in the region of the torso of the patient is examined, the movement of such tissue that is caused by cardiac activity or breathing of the patient should also be taken into account in the generation of images of this tissue in order to be able to acquire high-quality images of the tissue that are free of movement artifacts.

For example, in the imaging of the heart itself as the tissue to be examined, in order to avoid such movement artifacts in the reconstructed slice images and 3D images of the heart, in the reconstruction of slice images and 3D images that takes place based on the acquired 2D x-ray projections of the heart, it is always sought to use only those 2D x-ray projections that have been acquired in the cardiac phase of the cardiac cycle of the patient in which the heart has performed practically no movement. It is typical to record an electrocardiogram (EKG) of the heart of the patient to determine the cardiac cycle of the heart of the patient.

For the generation of slice images and 3D images of the heart, 2D x-ray projections of the heart are normally acquired with parallel recording of the electrocardiogram over multiple cardiac cycles, and only thereafter is a selection made as to the 2D projections that are suitable for the reconstruction (based on the electrocardiogram). For this reason, this type of method is called a retrospective method.

In an alternative procedure, 2D x-ray projections of the heart are likewise acquired over multiple cardiac cycles, but based on an electrocardiogram acquired in parallel, acquisition of the respective projections takes place only when the heart is located in a cardiac phase at which it performs practically no movement. This procedure has the advantage that the patient is exposed to a lower dose of x-ray radiation.

If the patient has a low and uniform heart rate i.e., a uniform cardiac cycle, such as below 60 bpm (beats per minute), a relatively short time period around the 60% position of the RR interval can be identified by analysis of the RR interval in the EKG, in which time period 2D x-ray projections can be acquired in each cardiac cycle, for example. In this context, a “pulsing window” is established, which is a time window in which x-ray radiation is applied. Generally, it is preferable for the pulsing window to substantially coincide with the rest phase of the heart. Under such circumstances, a low dose of x-ray radiation for the acquisition of 2D x-ray projections from different projection directions that is required for the reconstruction of images of said heart is applied to the patient. A high image quality is simultaneously achieved.

The imaging procedure also can be implemented under consideration of breathing movements. A breathing belt is frequently used that embodies a motion sensor and is placed on the patient in the chest area to detect breathing movements. The detected breathing cycle is taken into account in the imaging.

### SUMMARY OF THE INVENTION

An object of the invention is based on the object to provide an installation, a method, and a data storage medium of the aforementioned type such that the operation of a medical apparatus can be controlled in an alternative manner.

According to the invention, this object is achieved by a method to control a medical apparatus of an installation having a contact device for a patient, into which contact device at least one electrical potential sensor that can be coupled to the body of the patient is integrated, a signal evaluation device to which the measurement signals generated with the at least one electrical potential sensor are supplied for evaluation. The medical apparatus which is connected with the signal evaluation device, in which medical apparatus measurement signals that relate to breathing activity and/or cardiac activity of the patient are acquired with the at least one electrical potential sensor coupled to the body of the patient upon contact of the patient with the contact device. Trigger signals are generated with the signal evaluation device based on the measurement signals that relate to the breathing cycle and/or the cardiac cycle of the patient. The operation of the medical apparatus is controlled based on the trigger signals.

The invention proceeds from the consideration that the use of conventional EKG electrodes is often laborious and uncomfortable for the patient. At least for male patients, the chest region must thus first be prepared by a partial removal of hair to apply the EKG electrodes. The use of an adhesive or contact agent is most often required to arrange the EKG electrodes on the skin. Furthermore, a certain dependency of the EKG signals with regard to the individual impedance of the skin of the patient occurs given the use of EKG electrodes.

Therefore, the conventional EKG electrodes that are to be galvanically coupled to the body of the patient foregone, and instead, in accordance with the invention, at least one potential sensor is arranged in a contact device. To execute the proposed method, the at least one potential sensor brought into contact with the body of the patient via the contact device and is coupled in this way to the body of the patient to acquire measurement signals. The contact of the potential sensor with the patient thus can take place indirectly (i.e. the patient can wear clothing).

If the potential sensor is coupled to the body of the patient in the region of the chest, measurement signals that relate to, characterize or identify the breathing and/or cardiac activity of the patient can be generated with the potential sensor. The operation of a medical apparatus can be controlled or influenced with trigger signals derived from the measurement signals.

According to an embodiment of the invention, the medical apparatus is an imaging medical apparatus with which, based on the trigger signals, apparatus image information of the patient can be acquired, in particular in the region of the breast or in the region of the abdomen of the patient.

According to a further embodiment of the invention, the medical apparatus is a radiation therapy apparatus, with which the radiation treatment of a tissue of the patient is controlled based on the trigger signals.

According to another embodiment of the invention, the at least one electrical potential sensor has at least two (advantageously three) electrodes that can be capacitively coupled to the body of the patient, with which electrodes measurement signals are generated in the form of difference measurement signals that pertain to the breathing and/or cardiac activity of the patient, and that are supplied to the signal evaluation device. Two of the three electrodes are active electrodes, while the third electrode represents a reference electrode for the two active electrodes. The difference measurement signals of the at least one potential sensor are generated based on the signals of the two active electrodes. The potential sensor normally also includes structural elements for signal processing, such as an instrumental amplifier, filter, an A/D converter, etc.

According to another embodiment of the invention, the measurement signals, in particular the difference measurement signals, are evaluated by the signal evaluation device by means of a Fourier and/or wavelet analysis in order to generate trigger signals that pertain to the breathing cycle and/or the cardiac cycle of the patient. Those measurement signals, or those signal portions of the measurement signals (including their signal energy) whose frequency is (for example) within the frequency bandwidth that is associated with a human heart (approximately 60 to 140 beats per minute), can be identified with the use of the Fourier and/or wavelet analysis. The measurement signals that are to be associated with the breathing of the patient can be identified in the same manner.

In an embodiment of the invention, the contact device is a belt to be arranged on or attached to the chest of the patient, in which belt is integrated or on which belt is arranged at least one electrical sensor. Such a belt is normally of elastic design in order to ensure a good contact of the potential sensor with the body surface of the patient.

In another embodiment of the invention the contact device is a patient support plate of a patient support table or a placement mat that can be arranged on the patient support plate of a patient support table, wherein the patient support plate or the placement mat has at least one electrical potential sensor that can be coupled to the body of the patient, the measurement signals of which electrical potential sensor are supplied to the signal evaluation device. The coupling of the at least one electrical potential sensor to the body of the patient takes place via the support of the patient on the patient support plate or on the placement mat.

In an embodiment of the invention the patient support plate or the placement mat has a number of electrical potential sensors that can be coupled to the body of the patient, which electrical potential sensors are arranged in a two-dimensional matrix and whose measurement signals are supplied to the signal evaluation device.

The use of a number of potential sensors, and in particular their arrangement in an array or, respectively, a two-dimensional matrix, simplifies the support of the patient on the patient support plate or placement mat since it does require special care to ensure that the chest region of the patient is located over a specific potential sensor. Furthermore, a number of measurement signals originating from various potential sensors are now available, so that those measurement signal that are or appear to be best suited for the generation of trigger signals can be selected.

According to an embodiment of the invention, the signal evaluation device has a computer and a multiplexer, and the measurement signals originating from the electrical potential sensors are supplied by the multiplexer to the computer. The measurement signals are accordingly processed in a time multiplexing method, and the measurement signals can be limited to those signals, or those signals can be selected that are relevant to the generation of trigger signals. These are normally the measurement signals with the largest amplitude values that originate from the electrical potential sensors that are arranged close to the heart of the patient, for example.

According to a further embodiment of the invention, the position of the heart of the patient in relation to the patient support plate or the placement mat is determined with the signal evaluation device, for example based on the signal strength or the signal amplitude of the relevant measurement signals, or the relevant signal portions of the measurement signals of the electrical potential sensors. The position of the heart of the patient in relation to the patient support plate or the placement mat is advantageously based on a cross-correlation analysis of measurement signals or, respectively, signal portions of measurement signals that originate from electrical potential sensors that are adjacent to one another. The determination of the position of the heart enables those electrical potential sensors or the measurement signals or signal portions originating from these electrical potential sensors (with which the cardiac activity can be detected or registered best) to be identified or selected even more precisely. The goal is a qualitatively high-grade detection of the cardiac cycle of the patient (including the QRS complex) in order to be able to derive suitable trigger signals to control the medical apparatus.

The determination of the attitude of the heart in relation to the support plate or the placement mat additionally has the advantage that those electrical potential sensors can be better identified or localized whose measurement signals are best suited for determination of the breathing cycle of the patient. Those electrical potential sensors whose measurement signals pertain to the chest breathing and those electrical potential sensors whose measurement signals pertain to abdominal breathing can thereby additionally be identified or localized. Variations in the breathing cycle can be determined and accounted for in this way depending on the region of the torso of the patient to generate suitable trigger signals.

According to an embodiment of the invention, the attitude or the alignment of the patient on the patient support plate or the placement mat and/or the section of the body of the patient in relation to the patient support plate or the placement mat is determined with the signal evaluation device based on the determination of the position of the heart in relation to said patient support plate or the placement mat, an image information is acquired for imaging the heart of the patient. In this way, for the imaging not only the alignment of the patient but also the body region of the patient that is to be scanned or sampled in the course of the imaging is determined automatically. This information can be directly used for the imaging in the imaging apparatus without this having to be acquired first with an overview scan (in the case of computed tomography).

According to a further embodiment of the invention, the size of the patient, the attitude of at least one arm, the attitude of at least one leg, the attitude of the torso and/or the attitude of the head of the patient in relation to the patient support plate or the placement mat are determined with the signal evaluation device, based on the measurement signals.

The determination of this information preferably takes place with the cooperation of the patient by the patient moving the corresponding body parts as instructed, and therefore measurement signals are generated that can be evaluated. However, even in the case of less cooperative patients, corresponding measurement signals can be generated by forced movements of the corresponding body parts, for example by a forced vibration of the table.

Furthermore, movements of the at least one arm, the at least one leg, the torso and/or the head of the patient in relation to the patient support plate or the placement mat can be determined with the signal evaluation device based on the measurement signals and be taken into account (in the imaging, for example). For example, for image acquisitions of the head of the patient with a magnetic resonance apparatus, which takes a long time, movements of the head can be detected and corrections can thus be implemented in the imaging.

According to a further embodiment of the invention, the attitude of various internal organs or various tissues of the patient in relation to the patient support plate or the placement mat is determined or estimated with the signal evaluation device, based on the knowledge about the attitude of the patient, in particular about the attitude of the head, the torso, the heart, the arms and the legs in relation to the patient support plate or the placement mat. Based on this information, various body segments of the patient are established in relation to the patient support plate or the placement mat, in which body segments image information must respectively be acquired for an imaging of an internal organ or a tissue of the patient. In imaging medical apparatuses, this information can be used for what is known as the “auto-align function,” in which image information in the body segment including the tissue is determined automatically depending on the tissue to be examined and based on the determined information about the attitude of the tissue in relation to the patient support plate or the placement mat.

The above object also is achieved according to the invention by an installation having a contact device for a patient, into which is integrated; at least one electrical potential sensor that can be coupled to the body of the patient, a signal evaluation device to which the measurement signals generated with the at least one electrical potential sensor are supplied for evaluation, a medical apparatus connected with the signal evaluation device, and a computer in which a computer program runs that causes one or more of the embodiments of the method described in the preceding to be executed.

According to embodiments of the invention, the contact device is a belt, a patient support plate of a patient support table, or a placement mat for a patient support table.

The medical apparatus can be a computed tomography apparatus, a C-arm x-ray apparatus, a PET apparatus, a SPECT apparatus, a magnetic resonance apparatus, or a radiation therapy apparatus.

The above object also is achieved in accordance with the present invention by a non-transitory, computer-readable data storage medium encoded with programming instructions that, when the data storage medium is loaded into a computerized signal evaluation device, cause the signal evaluation device to implement one or more embodiments of the above-described method.

### BRIEF DESCRIPTION OF THE DRAWINGS

FIG. 1 illustrates a medical apparatus in accordance with the invention in the form of a computed tomography apparatus.

FIG. 2 shows the patient support plate of the computed tomography apparatus of FIG. 1, with a number of integrated electrical potential sensors arranged in a two-dimensional matrix.

FIG. 3 shows the basic design of an electrical potential sensor.

FIG. 4 shows a medical apparatus in accordance with the invention in the form of a radiation therapy apparatus.

FIG. 5 shows a patient support plate or a placement mat with only one electrical potential sensor.

FIG. 6 shows the computed tomography apparatus of FIG. 1, in an embodiment wherein a belt with an electrical potential sensor is placed on the patient.

### DESCRIPTION OF THE PREFERRED EMBODIMENTS

Identical or functionally identical elements in figures are provided throughout with the same reference characters. The representations in figures are schematic and not necessarily true to scale. In the exemplary embodiments of the invention, the medical apparatuses are a computed tomography apparatus and a radiation therapy apparatus which are discussed in the following and without limitation of the invention only insofar as is deemed necessary for comprehension of the invention.

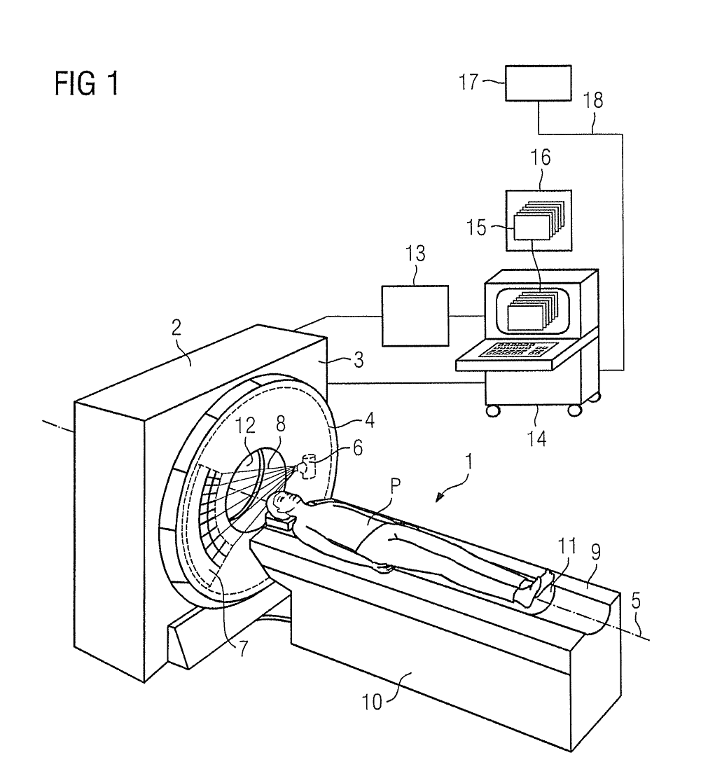

The computed tomography apparatus 1 shown in FIG. 1 has a gantry 2 with a stationary part 3 and with a schematically indicated part 4 that can be rotated around a system axis 5. The part 4 is borne by means of a support (not shown in FIG. 1) such that it can rotate relative to the stationary part 3. In the exemplary embodiment of the invention, the rotatable part 4 has an x-ray system formed by an x-ray source 6 and an x-ray radiation detector 7 that are arranged opposite one another at the rotatable part 4. In the operation of the computed tomography apparatus 1, x-ray radiation 8 emanates from the x-ray source 6 in the direction of the x-ray radiation detector 7, penetrates a measurement subject and is detected by the x-ray radiation detector 7 in the form of detector measurement data or detector measurement signals.

The computed tomography apparatus 1 furthermore has a patient bed 9 to support a patient P to be examined. The patient bed 9 has a bed base 10 on which is arranged a patient support plate 11 provided to actually support the patient P. The patient support plate 11 can be displaced in a motorized fashion in the direction of the system axis 5 relative to the bed base 10 such that it, together with the patient P, can be introduced into the opening 12 of the gantry 2 for the acquisition of 2D x-ray projections of the patient P, for example in a spiral scan.

The computational processing of the 2D x-ray projections acquired with the x-ray system or, respectively, the reconstruction of slice images, 3D images or a 3D data set based on the detector measurement data or the detector measurement signals of the 2D x-ray projections takes place with an image computer 13 (schematically presented) of the computed tomography apparatus 1.

The computed tomography apparatus 1 has a computer 14 with which computer programs can be and are executed to operate and control the computed tomography apparatus 1. The computer 14 does not need to be designed as a separate computer 14, but can be integrated into the computed tomography apparatus 1.

In the exemplary embodiment of the invention, a computer program 15 that realizes the method according to the invention to control a medical apparatus (presently the computed tomography apparatus 1) is loaded into the computer 14. The computer program 15 represents a special operating mode (among others) for the computed tomography apparatus 1 and can have been loaded into the computer 14 from a portable data medium (from a CD 16 or from a memory stick, for example) or from a server 17 via a network 18 (which can be a public network and also a network internal to the clinic or hospital).

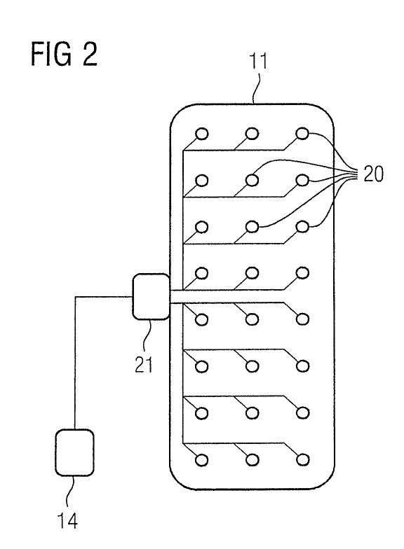

In the exemplary embodiment of the invention, a number of electrical potential sensors 20 that are arranged in a two-dimensional matrix is integrated as a contact device for the patient P into the patient support plate 11. FIG. 2 shows in a schematic view, the arrangement of the electrical potential sensors 20 inside the patient support plate 11. The arrangement of the electrical potential sensors 20 inside the patient support plate 11 is such that a coupling of the electrical potential sensors 20 to the body surface of the patient P takes place upon placement of the patient P on the patient support plate 11, such that measurement signals can be generated with the electrical potential sensors 20.

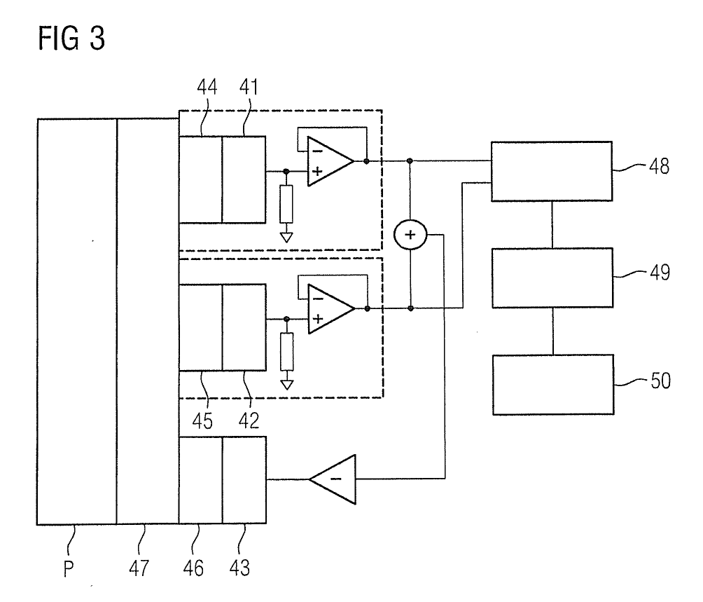

FIG. 3 shows the principle design of one of the electrical potential sensors 20 that is used in the case of the present exemplary embodiment of the invention. The electrical potential sensor 20 includes three electrodes 41 through 43 that can be, or presently already are, coupled capacitively to the body of the patient P, of which three electrodes 41 through 43 the electrodes 41 and 42 are active electrodes. The electrode 43 is a reference electrode or what is known as a “driven ground plane”. All three electrodes are provided at the patient with an insulating layer 44 through 46 and are customarily coupled to the body of the patient P across the clothing of the patient P. Difference measurement signals are generated based on the signals of the two active electrodes 41, 42.

For the present invention, it is primarily the dynamic distance variation between the body surface of the patient P and the electrodes of the electrical potential sensors 20 due to the cardiac activity of the patient P as well as the rise and fall of the ribcage as a result of breathing of the patient P that are relevant.

In the exemplary embodiment of the invention, each electrical potential sensor 20 also has electrical structural elements for signal pre-processing. The signals of the active electrodes 41, 42 are thus supplied to an instrument preamplifier 48. Furthermore, filters 49 as well as an A/D converter 50 can be provided. However, the electrical potential sensors 20 do not necessarily need to have such structural elements or all cited structural elements for signal pre-processing or for signal processing. Insofar as it is feasible in terms of measurement technology, the signals of the active electrodes can also first be directed out of the patient support plate 11 and then be processed further.

In the exemplary embodiment of the invention, the electrical potential sensors 20 are connected with a signal evaluation device that has a multiplexer 21 and a computer to evaluate the difference measurement signals. In the case of the present exemplary embodiment of the invention, the computer 14 forms the computer of the signal evaluation device. The difference measurement signals of the electrical potential sensors 20 are supplied to the computer 14 via the multiplexer 21.

The computer 14 evaluates the difference measurement signals received from the multiplexer 21, wherein in the case of the present exemplary embodiment of the invention it subjects the difference measurement signals of each electrical potential sensor 20 to a Fourier and/or a wavelet analysis in order to in particular initially identify those electrical potential sensors 20 of the matrix whose difference measurement signals or whose signal portions of the difference measurement signals have a signal energy that is typical of cardiac activity and a frequency that lies within the frequency bandwidth that is associated with a human heart (approximately 60 to 140 beats per minute).

The position of the heart of the patient P in relation to the patient support plate 11 is determined via the electrical potential sensors 20 that are identified in such a manner. In the case of the present exemplary embodiment of the invention, a cross-correlation analysis of the difference measurement signals which originate from identified adjacent electrical potential sensors 20 additionally takes place in order to determine the precise position of the heart of the patient P in relation to the patient support plate 11.

The activity of the heart of the patient is determined based on the analysis of the difference measurement signals of the identified electrical potential sensors 20 arranged near the heart of the patient P. Ideally, the cardiac cycle of the patient P or, respectively, an electrocardiogram of the heart of the patient P is determined so that trigger pulses to establish an aforementioned “pulsing windows” can be generated based on the determined cardiac cycle or, respectively, the electrocardiogram. For example, in this way the acquisition of x-ray projections of the chest region (in particular of the heart of the patient P) can be controlled, meaning that x-ray projections in which the heart of the patient P makes practically no movement are acquired only during the “pulsing window” established by the trigger pulses.

If the attitude of the heart in relation to the patient support plate 11 is determined, those electrical potential sensors 20 whose difference measurement signals are best suited to determine the breathing cycle of the patient P can moreover be better identified or located. In particular, those electrical potential sensors 20 whose difference measurement signals pertain to chest breathing and those electrical potential sensors 20 whose difference measurement signals pertain to diaphragmatic breathing can be identified or, respectively, located.

The breathing cycle pertaining to chest breathing can inasmuch be determined based on the identified electrical potential sensors 20 whose difference measurement signals pertain to the chest breathing. Ultimately, trigger signals with which at least one time period of the breathing cycle is established for acquisition of x-ray projections of the chest region of the patient P (in particular of the lungs of the patient) can be generated using the breathing cycle pertaining to chest breathing.

The breathing cycle pertaining to the diaphragmatic breathing can be determined in a comparable manner based on the identified electrical potential sensors 20 whose difference measurement signals pertain to the diaphragmatic breathing. Ultimately, trigger signals with which at least one time period of the breathing cycle for acquisition of acquisition projections of the region of the abdomen of the patient P is established can be generated using the breathing cycle pertaining to the abdominal breathing.

With regard to the computed tomography apparatus 1, the respective determined or established trigger signals can be used both for the prospective image generation method that was already described—in which x-ray projections are only acquired when optimally no movement of the torso of the patient P takes place, which movement is inherently caused by the cardiac and/or breathing activity—and for a retrospective image generation method in which, after the acquisition of the x-ray projections based on the trigger signals, those x-ray projections that were acquired at a phase in which optimally no movement of the torso of the patient that was caused by the cardiac and/or breathing activity existed are selected for an image reconstruction.

The difference measurement signals of the electrical potential sensors 20 can furthermore be used to determine the alignment, the size, the attitude of at least one arm, the attitude of at least one leg, the attitude of the torso and/or the attitude of the head of the patient P in relation to the patient support plate 11. This preferably takes place with the cooperation of the patient P in that said patient P makes corresponding movements of the corresponding body parts so that defined difference measurement signals are generated whose evaluation on the part of the computer 14 supplies the desired information.

In the case of the exemplary embodiment of the invention, based on the obtained information about the size, the alignment, the attitude of the heart, the head, the arms and the legs of the patient P in relation to the patient support plate 11 the attitude of various internal organs (such as the attitude of the lungs, of the intestine etc.) or various other tissues of the patient P (such as the spinal column, the pelvis etc.) are determined in relation to the patient support plate 11, stored, and based on this—and under consideration of the known attitude and position of the patient support plate 11 and the gantry 2 relative to one another—various body segments or scan segments of the patient P can be established or defined in relation to the patient support plate 11 and stored, in which segments image information must respectively be acquired for an imaging of an internal organ or a tissue of the patient P. A function known as an “auto-align function” is thus achieved. If the heart of the patient P should be scanned, the scan region—thus the region in which x-ray projections of the heart must be acquired from different projection directions during rotation of the x-ray system around the system axis 5—is already established or, respectively, defined, and does not need to first be determined by means of an overview scan. The same is true for the other organs and tissue of the patient P.

Furthermore, movements of the patient, such as movements of an arm, a leg, the torso or the head of the patient P in relation to the patient support plate 11,—can be determined based on the difference measurement signals and the computer 14 (in particular during an acquisition of x-ray projections), and movement artifacts can be avoided in the reconstructed images of a tissue of the patient P under consideration of the determined movements.

The computed tomography apparatus 1 can be used not only for imaging but also for planning of procedures (or also to plan a radiation therapy) in order to correlate the movement of a tissue of a patient that is to be therapeutically treated, for example with the breathing phases of said patient.

The imaging medical apparatus can moreover also be a C-arm x-ray apparatus, a PET apparatus, a SPECT apparatus or a magnetic resonance apparatus.

For a use of the electrical potential sensors in a magnetic resonance apparatus, these can also be produced from a non-magnetic metal.

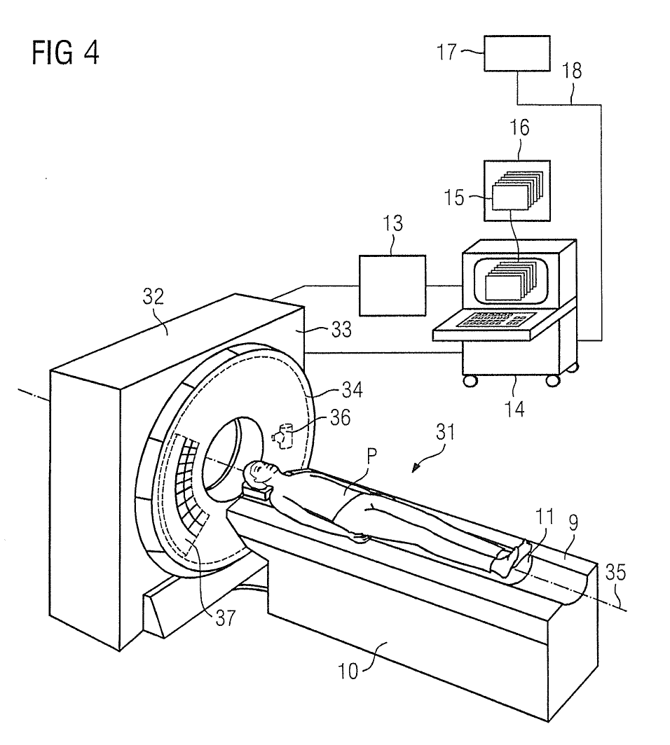

Moreover, the medical apparatus can also be a radiation therapy apparatus. FIG. 4 shows such a radiation therapy apparatus 31 in a significantly schematic presentation, which apparatus 31 comprises a gantry 32 with a stationary part 33 and with a schematically indicated part 34 that is rotatable around a system axis 35, which part 34 is borne by means of a support (not shown in FIG. 4) such that it can rotate relative to the stationary part 33. The rotatable part 34 has a therapeutic x-ray source 36 and an x-ray detector 37 arranged opposite this for MeV imaging. The remaining components of the radiation therapy apparatus 31 (such as the patient bed 9, etc.) essentially correspond to the components of the computed tomography apparatus 1, which is why these are provided with the same reference characters. The therapeutic x-ray source 36 serves to charge a tissue of the patient P that is to be treated therapeutically with therapeutic x-rays that have a photon energy in the MeV range.

In the case of a radiation therapy apparatus, the trigger signals generated from the difference measurement signals of the electrical potential sensors 20 of the patient support plate 11 are used to charge the tissue of the patient P with the therapeutic x-ray radiation only when optimally no movement (caused by the cardiac or breathing activity of the patient P) of the tissue to be therapeutically treated is present and/or when the tissue to be therapeutically treated is located in a defined therapy position, such that tissue that is not to be therapeutically treated is not also charged with x-ray radiation.

In contrast to the described exemplary embodiments of the invention, the electrical potential sensors do not necessarily need to be integrated into the patient support plate. The possibility also exists to arrange the electrical potential sensors in a placement mat that can be or, respectively, is placed on the patient support plate. This is particularly advantageous for already existing medical apparatuses that can simply be retrofitted in this manner.

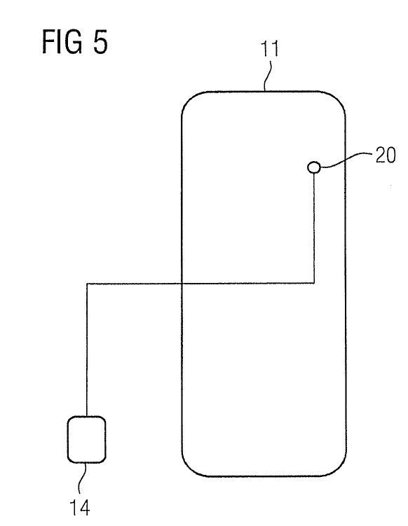

Also, a matrix of electrical potential sensors does not necessarily need to exist. Insofar as it is appropriate, only one electrical potential sensor can also be presented in a patient support plate or placement mat. Using the patient support plate 11, FIG. 5 illustrates this simplified design in which only one electrical potential sensor 20 is present. In this case, no multiplexer is required.

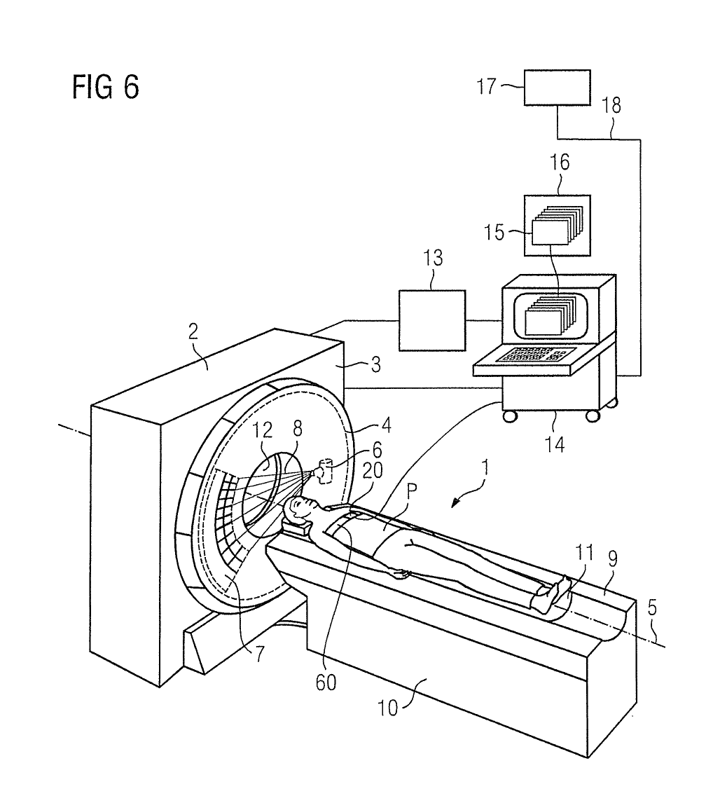

Alternatively, at least one electrical potential sensor 20 can also be arranged in or be integrated into a belt 60 that is placed on the chest of a patient P. FIG. 6 shows this embodiment of the invention according to FIG. 1. In this case, the patient support plate 11 does not need to have any electrical potential sensors. The belt is normally elastic in order to ensure a good contacting of the electrical potential sensor 20 with the body of the patient P.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method to control a medical apparatus of a medical installation, said medical apparatus having an interior opening in which an interaction region of the medical apparatus is located, in which radiation is emitted into a patient in the opening or radiation is detected from a patient in the opening, said method comprising:
placing a clothed patient on a movable patient support surface in said medical apparatus, said patient support surface being selected from the group consisting of a patient support plate of a patient support table, and a placement mat configured to be arranged on a patient support plate of a patient support table, said patient support surface comprising a plurality of electrical potential sensors integrated therein to form a two-dimensional signal matrix, that each interact with the body of the clothed patient, each electrical potential sensor being comprised of at least three electrodes that are each capacitively coupled to the patient;
with at least one electrical potential sensor in said plurality of electrical potential sensors coupled to the body of the patient, generating a measurement signal that represents a physiological activity of the clothed patient selected from the group consisting of breathing activity and cardiac activity, by operating two of said electrodes of said at least one electrical potential sensor as active electrodes and a third of said electrodes of said at least one electrical potential sensor as a reference electrode with respect to said active electrodes, in order to sense a dynamic distance variation between a body surface of the patient and said active electrodes;
in a signal evaluation processor supplied with said measurement signal, evaluating said measurement signal to obtain an evaluation result and, from said evaluation result, generating trigger signals;
in said signal evaluation processor, also determining a position of the heart of the patient, with respect to the patient support bed, by evaluating respective measurement signals from multiple electrical potential sensors in said plurality of electrical potential sensors;
in said signal evaluation processor, from the position of the heart of the patient that has been determined with respect to said patient support surface, also determining at least one of a positional attitude of the patient on the patient support surface, an alignment of the patient on the patient support surface, and a portion of the body of the patient with respect to the patient support surface;
from said signal evaluation processor, controlling movement of said patient support surface, with said clothed patient thereon, in order to position said clothed patient on said patient support surface relative to said interaction region so as to cause a selected anatomical portion of the clothed patient to be situated in said interaction region; and
from said signal evaluation processor, controlling operation of said medical apparatus based on said trigger signals to emit radiation or detect radiation in a timed relation to said physiological activity while said selected anatomical portion of the clothed patient is situated in said interaction region.

2. A method as claimed in claim 1 comprising, in said signal evaluation processor, generating said measurement signal that represents said physiological activity of the patient as a difference between signals respectively obtained between said active electrodes and said reference electrode.

3. A method as claimed in claim 1 comprising, in said signal evaluation processor, evaluating said measurement signal of said at least one electrical potential sensor by implementing an analysis selected from the group consisting of Fourier analysis and wavelet analysis, in order to obtain said evaluation result.

4. A method as claimed in claim 1 comprising multiplexing respective measurement signals from said multiple electrical potential sensors for supply to said processor.

5. A method as claimed in claim 1 comprising, in said signal evaluation processor, determining the position of the heart of the patient with respect to the patient support device by implementing a cross-correlation analysis of respective measurement signals originating respectively from electrical potential sensors, among said multiple electrical potential sensors, that are adjacent to each other.

6. A method as claimed in claim 1 wherein said medical apparatus is a medical imaging apparatus, and comprising, from said signal evaluation processor, controlling said operation of said imaging medical apparatus based on said trigger signals in order to operate the medical imaging apparatus to acquire image data from said anatomical portion of the clothed patient situated in said interaction region.

7. A method as claimed in claim 6 comprising selecting said medical imaging apparatus from the group consisting of x-ray computed tomography (CT) apparatuses, magnetic resonance tomography (MRT) apparatuses, positron emission tomography (PET) apparatuses, and single-photon emission computed tomography (SPECT) apparatuses.

8. A method as claimed in claim 1 wherein said medical apparatus is a radiation therapy apparatus, and comprising, from said signal evaluation processor, controlling said operation of said radiation therapy apparatus based on said trigger signals in order to cause said radiation therapy apparatus to administer radiation therapy to said selected anatomical portion of the clothed patient situated in said interaction region.

9. A medical installation comprising:
a medical apparatus having an interior opening in which an interaction region of the medical apparatus is located, in which radiation is emitted into a patient in the opening or radiation is detected from a patient in the opening;
a movable patient support surface in said medical apparatus, said patient support surface being selected from the group consisting of a patient support plate of a patient support table, and a placement mat configured to be arranged on a patient support plate of a patient support table, said patient support surface being adapted to receive a clothed patient thereon and said patient support surface comprising a plurality of electrical potential sensors integrated therein to form a two-dimensional signal matrix, that each interact with the body of the clothed patient, each electrical potential sensor being comprised of at least three electrodes that are each capacitively coupled to the patient;
at least one electrical potential sensor in said plurality of electrical potential sensors coupled to the body of the patient being configured to generate a measurement signal that represents a physiological activity of the clothed patient selected from the group consisting of breathing activity and cardiac activity, with two of said electrodes of said at least one electrical potential sensor operated as active electrodes and a third of said electrodes of said at least one electrical potential sensor operated as a reference electrode with respect to said active electrodes, in order to sense a dynamic distance variation between a body surface of the patient and said active electrodes;
a signal evaluation processor supplied with said measurement signal, configured to evaluate said measurement signal to obtain an evaluation result and, from said evaluation result, to generate trigger signals;
said signal evaluation processor being configured to also determine a position of the heart of the patient, with respect to the patient support bed, by evaluating respective measurement signals from multiple electrical potential sensors in said plurality of electrical potential sensors;
said signal evaluation processor, being also configured to determine, from the position of the heart of the patient that has been determined with respect to said patient support surface, at least one of a positional attitude of the patient on the patient support surface, an alignment of the patient on the patient support surface, and a portion of the body of the patient with respect to the patient support surface;
said signal evaluation processor being configured to control movement of said patient support surface, with said clothed patient thereon, in order to position said clothed patient on said patient support surface relative to said interaction region so as to cause a selected anatomical portion of the clothed patient to be situated in said interaction region; and
said signal evaluation processor being configured to control operation of said medical apparatus based on said trigger signals to emit radiation or detect radiation in a timed relation to said physiological activity while said selected anatomical portion of the clothed patient is situated in said interaction region.

10. A medical installation as claimed in claim 9 wherein said processor is configured to generate said measurement signal that represents said physiological activity of the patient as a difference between signals respectively obtained between said active electrodes and said reference electrode.

11. A medical installation as claimed in claim 9 wherein said signal evaluation processor is configured to evaluate said measurement signal of said at least one electrical potential sensor by implementing an analysis selected from the group consisting of Fourier analysis and wavelet analysis, in order to obtain said evaluation result.

12. A medical installation as claimed in claim 9 comprising a multiplexor that multiplexes respective measurement signals from said multiple electrical potential sensors for supply to said processor.

13. A medical installation as claimed in claim 9 wherein said signal evaluation is configured to determine the position of the heart of the patient with respect to the patient support device by implementing a cross-correlation analysis of respective measurement signals originating respectively from electrical potential sensors, among said multiple electrical potential sensors, that are adjacent to each other.

14. A medical installation as claimed in claim 9 wherein said medical apparatus is a medical imaging apparatus, and wherein said signal evaluation processor is configured to control said operation of said medical imaging apparatus based on said trigger signals in order to operate the medical imaging apparatus to acquire image data from said anatomical portion of the clothed patient situated in said interaction region.

15. A medical installation as claimed in claim 14 wherein said medical imaging apparatus is selected from the group consisting of x-ray computed tomography (CT) apparatuses, magnetic resonance tomography (MRT) apparatuses, positron emission tomography (PET) apparatuses, and single-photon emission computed tomography (SPECT) apparatuses.

16. A medical installation as claimed in claim 9 wherein said medical apparatus is a radiation therapy apparatus, and wherein said signal evaluation processor is configured to control said operation of said radiation therapy apparatus based on said trigger signals in order to cause said radiation therapy apparatus to administer radiation therapy to said selected anatomical portion of the clothed patient situated in said interaction region.
